(19) United States
(12) Patent Application Publication
(10) Pub. No.: US 2022/0387990 A1
(43) Pub. Date: Dec. 8, 2022

(54) BIOAGENT IDENTIFICATION THROUGH OPTICAL SURFACE PROFILING IN CONJUNCTION WITH A SUITABLE MACHINE LEARNING MODEL

(71) Applicant: THE PENN STATE RESEARCH FOUNDATION, University Park, PA (US)

(72) Inventors: Yin-Ting Yeh, University Park, PA (US); Mauricio Terrones, State College, PA (US)

(21) Appl. No.: 17/755,019

(22) PCT Filed: Nov. 3, 2020
(86) PCT No.: PCT/US2020/058659

§ 371 Date: Apr. 19, 2022

Related U.S. Application Data
(60) Provisional application No. 62/930,157, filed on Nov. 4, 2019.

(51) Int. Cl. B01L 3/00; G01J 3/44; G01N 21/65; G01N 21/64

(52) U.S. Cl. CPC B01L 3/5027; G01J 3/44; G01N 21/658; G01N 21/645

(57) ABSTRACT

Embodiments relate to a bioagent capture and identification system including a microfluidic platform for label-free, size-based capture, enrichment, and optical profiling of bioagents using vertically aligned carbon nanotubes coated in gold nanoparticles. Bioagent identification can be automated using machine learning. Captured bioagents remain viable after capture and analysis. In the nanotube fabrication process, catalyst precursor layers are fabricated using patterned stamps. In addition, nanotube diameter and density are increased by increasing the concentration of metal content in the catalyst precursor layer.

### CROSS-REFERENCE TO RELATED APPLICATIONS

This application is related to and claims the benefit of U.S. provisional application No. 62/930,157, filed Nov. 4, 2019, the entire contents of which is incorporated herein by reference.

### FIELD OF THE INVENTION

Embodiments relate to a bioagent capture and identification system including a microfluidic platform for label-free, size-based capture, enrichment, and optical profiling of bioagents using vertically aligned carbon nanotubes coated in gold nanoparticles.

### BACKGROUND OF THE INVENTION

Conventional methods and system for bioagent identification are limited in that they fail to provide adequate or sensitive detection by spectroscopy techniques. Conventional methods and systems also fail to provide a means for quick and accurate detection and identification of viruses without the use of labeling techniques.

Existing methods and systems for bioagent identification can be appreciated from US 2017/0038285, WO 2009/102783, WO 2012/031042, “Monodisperse Multiwall Carbon Nanotubes Obtained with Ferritin as Catalyst” from Institut de Physique des Nanostructures, Faculte' des Sciences de Base, Ecole Polytechnique Fe'de'rale de Lausanne, CH-1015 Lausanne EPFL, Switzerland, March 2006, by Jean-Marc Bonard et al., “Deep Convolutional Neural Networks for Raman Spectrum Recognition: A Unified Solution”, Aug. 18, 2017, by Jinchao Liu et al., and “Carbon Nanotube and Gold-Based Materials: A Symbiosis” from Chemistry, 2010, by Rajpal Singh et al.

### SUMMARY OF THE INVENTION

Embodiments relate to a bioagent capture and identification system including a microfluidic platform for label-free, size-based capture, enrichment, and optical profiling of bioagents using vertically aligned carbon nanotubes coated in gold nanoparticles. Bioagent identification can be automated using machine learning. Captured bioagents remain viable after capture and analysis. In the nanotube fabrication process, catalyst precursor layers are fabricated using patterned stamps. In addition, nanotube diameter and density are modulated by increasing or decreasing the concentration of metal content in the catalyst precursor layer.

The system provides for a multifunctional and portable platform for rapid virus capture and sensitive in-situ identification by Raman spectroscopy. The captured viruses are viable and enriched, thus providing effective sample preparation for existing standard methods for virus analysis, including cell culture for virus isolation, immuno-staining, and next-generation sequencing. The system can perform enrichment in just a few minutes after capture and achieves a sensitivity comparable to that of RT-qPCR with a 70˜90% accuracy.

In an exemplary embodiment, a bioagent capture and identification device includes a substrate and a vertically-aligned carbon nanotube (CN×CNT) array grown on the substrate, the CN×CNT array comprising a plurality of carbon nanotubes having an inter-tubular distance between each carbon nanotube. The substrate is patterned with a particle precursor. At least one nanotube is decorated with an enriching particle.

Some embodiments include a casing having an inlet and an outlet.

In some embodiments, the casing has a casing top, a casing bottom, and casing sides, and the CN×CNT array is vertically orientated with respect to the casing top and the casing bottom.

In some embodiments, the substrate and/or the CN×CNT array is/are bonded to at least a portion of the casing top and/or at least a portion of the casing bottom.

In some embodiments, patterning the substrate with a particle precursor causes a change in the inter-tubular distance between each carbon nanotube.

In some embodiments, the inter-tubular distance between two carbon nanotubes differs from the inter-tubular distance between two other carbon nanotubes.

In some embodiments, the inter-tubular distance between two carbon nanotubes varies along a length of the two carbon nanotubes.

In some embodiments, the particle precursor includes a Fe particle.

In some embodiments, decorating with the enriching particle functionalizes a carbon nanotube.

In some embodiments, the enriching particle includes an Au particle.

In some embodiments, a nanotube of the CN×CNT array is doped with a dopant.

In some embodiments, the dopant is nitrogen.

In some embodiments, at least one carbon nanotube has a herringbone shape.

In some embodiments, a portion of the casing is removable.

In some embodiments, the device is a microfluidic platform configured to receive a solution containing a species via the inlet, cause the solution to pass over or through the CN×CNT array, and expel the solution via the outlet. The inter-tubular distance between each carbon nanotube is tunable and selected to capture a species based on size.

In an exemplary embodiment, a bioagent capture and identification system includes a microfluidic device comprising a substrate and a vertically-aligned carbon nanotube (CN×CNT) array grown on the substrate, the CN×CNT array comprising a plurality of carbon nanotubes having an inter-tubular distance between each carbon nanotube. The substrate is patterned with a particle precursor. At least one nanotube is decorated with an enriching particle. The microfluidic device is configured to receive a solution containing a species and capture the species within the inter-tubular distance between two carbon nanotubes based on size. The system includes a spectrometer configured to generate spectra data of the captured species. The system includes a computer system comprising a computer device and a database, the computer system configured to receive the spectra data and use machine learning techniques to identify the species.

In some embodiments, the spectrometer is a Raman spectrometer.

In some embodiments, the database includes historical spectra data of different species.

In an exemplary embodiment, a method of fabricating a platform for a bioagent capture and identification device involves: patterning precursor particles onto the substrate; growing a vertically-aligned carbon nanotube (CN×CNT) array; and coating a nanotube of the CN×CNT array with an enriching particle.

In some embodiments, the method involves doping a nanotube of the CN×CNT array with a dopant.

Further features, aspects, objects, advantages, and possible applications of the present invention will become apparent from a study of the exemplary embodiments and examples described below, in combination with the Figures, and the appended claims.

### BRIEF DESCRIPTION OF THE DRAWINGS

The above and other objects, aspects, features, advantages and possible applications of the present invention will be more apparent from the following more particular description thereof, presented in conjunction with the following drawings, in which:

FIG. 1 shows an embodiment of the bioagent capture and identification system.

FIG. 2A shows an SEM image of an exemplary herringbone enriched-CNT structure, FIG. 2B shows an exemplary platform device, and FIG. 2C shows another exemplary platform device.

FIG. 3 shows an exemplary process flow for an embodiment of the bioagent capture and identification system.

FIGS. 4A-D show an exemplary fabrication process for fabricating an embodiment of the platform device.

FIG. 5A shows a TEM image of a CN×CNT, FIG. 5B shows Raman spectra with signature peaks of nitrogen-doped carbon nanotubes, FIG. 5C shows the height of the aligned CN×CNTs synthesis with different concentrations of precursors and synthesis v. growth time, FIG. 5D shows a histogram of gold nanoparticles decorated on CN×CNTs, and FIG. 5E shows a TEM and Energy-dispersive X-ray spectroscopy (EDX) of the CN×CNT being decorated with gold nanoparticles.

FIG. 6A shows a histogram of diameters of fluorescent particles, FIG. 6B shows a standard curve of fluorescence intensity as a function of particle concentrations; abs=absorbance, and FIGS. 6C-D show the capture efficiency of the platform device.

FIG. 7A shows Avian influenza virus H5N2 captured by an embodiment of the platform device and a histogram of H5N2 diameter and TEM image of H5N2, and FIG. 7B shows immunostaining of media without H5N2 (control).

FIG. 8 shows Raman spectra of cell culture supernatant and swab media without H5N2 virus (negative control).

FIG. 9A shows results of Dot-ELISA for CN×CNT structure without viruses through egg propagation, and FIG. 9B shows RT-qPCR results of H5N2 and 18S rRNA before and after enrichment by an embodiment of the disclosed method.

FIG. 10 shows an exemplary bioinformatic pipeline for sequencing data analysis and processing.

FIG. 11A shows a coverage plot of mapped avian influenza virus reading on genomic segments after capture and enrichment by an embodiment of the method disclosed herein for H5N2, and FIG. 11B shows a coverage plot of mapped avian influenza virus reading on genomic segments after capture and enrichment by an embodiment of the method disclosed herein for H7N2. HA=hemagglutinin; NA=neuraminidase; PB1, PB2, PA=polymerases; NP=nucleoprotein; M=matrix; NS=non-structural protein.

FIG. 12 shows images and histograms of sizes of Human viruses: Rhinovirus; influenza type A; and parainfluenza type 3.

FIG. 13 shows RT-qPCR results of respiratory viruses before and after capture by an embodiment of the method disclosed herein.

FIG. 14 shows coverages of influenza virus segments after virus capture and mapping of NGS reads to a reference strain (A/New York/03/2016(H3N2)). S001 represents the sample before enrichment, S007 represents the sample after one round of enrichment, and sample S013 represents the sample after two rounds of enrichment.

FIG. 15 shows normalized coverages of human parainfluenza type 3 genome after virus capture and mapping of NGS reads to reference strain MF973163. S006 is the unenriched sample, S012 is the sample following one enrichment step, and S018 is the sample following two enrichment steps.

FIGS. 16A-B show a design and working principle of VIRRION for effective virus capture and identification. FIG. 16 A is an illustration of (i) size-based capture and (ii) in-situ Raman spectroscopy for label-free optical virus identification. Images of electron microscopy showing captured avian influenza virus H5N2 by CN×CNT arrays. FIG. 16 B is an on-chip virus analysis and enrichment for next generation sequencing, (i) On-chip immunostaining for captured H5N2, (ii) On-chip viral propagation through cell culture, and (iii) Genomic sequencing and analysis of human parainfluenza type 3 (HPIV). track 1: scale of the base pair position; track 2: variant analysis by mapping to strain #MF973163, color code: deletion (black), transition (A-G, fluorescent green; G-A, dark green; C-T, dark red; T-C, light red), transversion (A-C, brown; C-A, purple; A-T, dark blue; T-A, fluorescent blue; G-T, dark orange; T-G, violet; C-G, yellow; G-C, light violet); track 3: coverage; track 4: regions of open reading frames.

FIGS. 17A-D show characterization of size-based capture. FIG. 17A is an illustration of capturing and separating three different sizes of fluorescently labelled silica particles by VIRRION having three zones of ITDs. FIG. 17B are a fluorescent and combined bright-field images of particles captured and separated by VIRRION into individual zones (scale bar is 200 μm). FIG. 17C shows capture efficiency of different silica particles captured by VIRRIONS under different flow rates. FIG. 17D shows capture efficiency of silica particles after multiple repeated captures by the same VIRRION.

FIGS. 18A-G show characterization of avian influenza virus captured and detected by VIRRION. FIG. 18A shows a process flow of VIRRION for avian influenza virus surveillance and discovery. FIG. 18B shows an SEM showing H5N2 virus particles captured by CN×CNT arrays. FIG. 18C shows Raman spectra of H5N2, H7N2, and Reovirus collected from VIRRION. FIG. 18D shows classification by PCA plot of Raman spectra collected from different avian viruses. FIG. 18E shows a process flow of virus identification by Raman spectroscopy with algorithm. FIG. 18F shows H5N2 virus propagated in embryonated chicken egg after viable capture and detection. FIG. 18G shows ratio of copy number of H5N2 and 18S rRNA before and after VIRRION enrichment.

FIGS. 19A-C show results using VIRRION for respiratory virus surveillance and discovery. FIG. 19A shows Raman spectra. FIG. 19B shows PCA plot of Raman fingerprint of the different viruses. Each dot represents a collected spectrum. FIG. 19C shows Circos plots of coverage and variants of captured influenza viruses. Genome segment sequencing and analysis of influenza A mapped to strain A/New York/03/2016 (H3N2). track 1: scale of the base pair position; track 2: variant analysis by mapping to strain H3N2 (KX413814-KX413821), color code: deletion (black), transition (A-G, fluorescent green; G-A, dark green; C-T, dark red; T-C, light red), transversion (A-C, brown; C-A, purple; A-T, dark blue; T-A, fluorescent blue; G-T, dark orange; T-G, violet; C-G, yellow; G-C, light violet); track 3: coverage; track 4: regions of open reading frame.

### DETAILED DESCRIPTION OF THE INVENTION

The following description is of an embodiment presently contemplated for carrying out the present invention. This description is not to be taken in a limiting sense, but is made merely for the purpose of describing the general principles and features of the present invention. The scope of the present invention should be determined with reference to the claims.

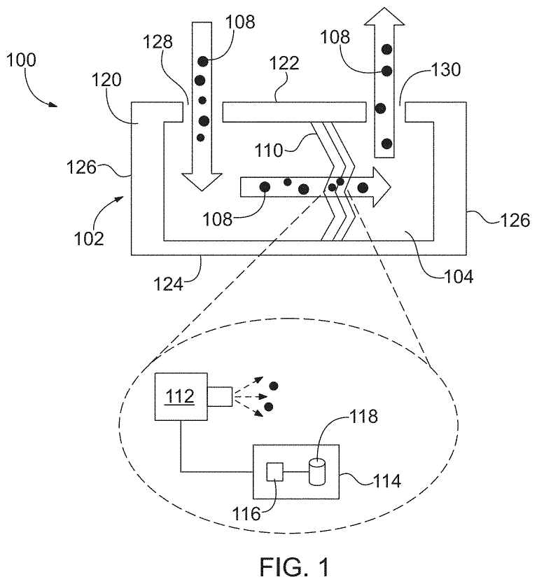

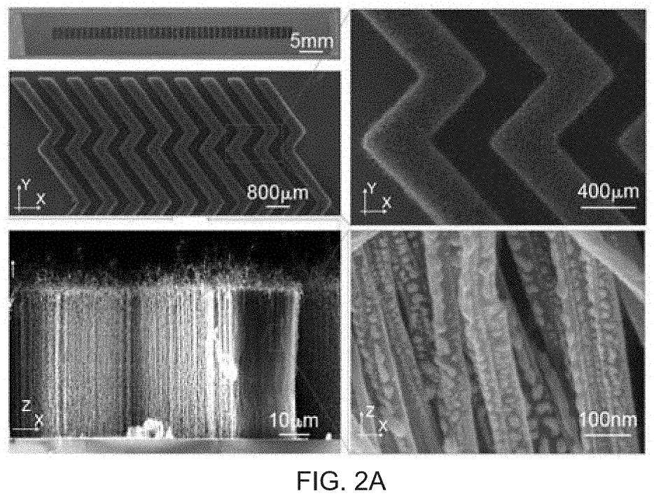

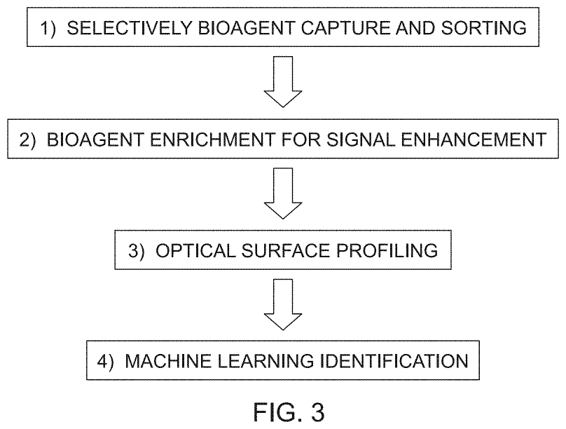

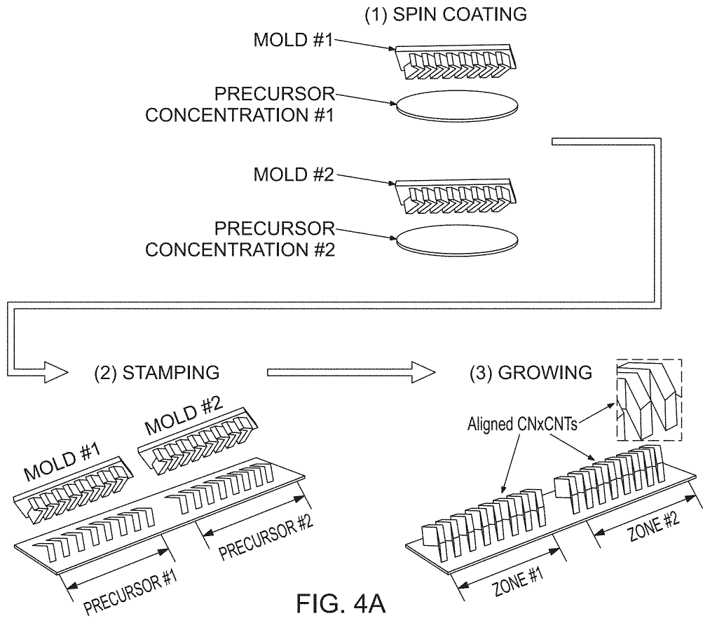

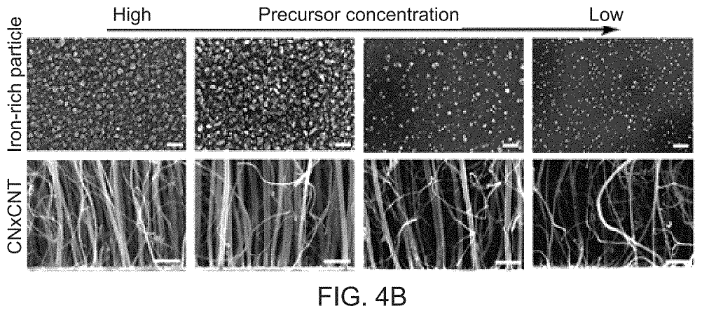

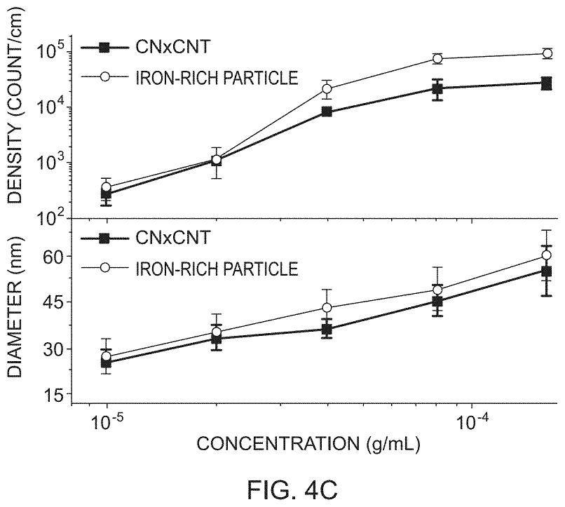

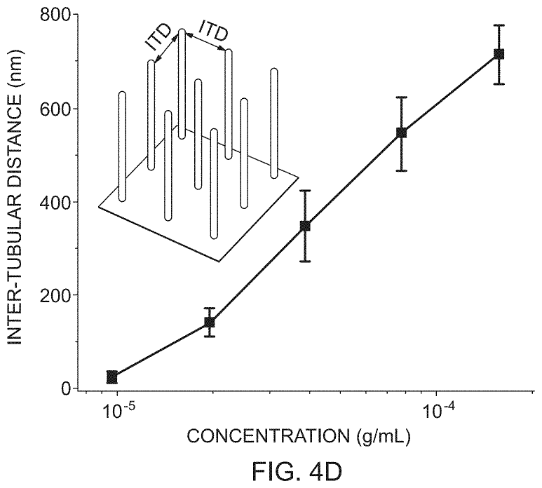

Referring to FIGS. 1-4, embodiments relate to a bioagent capture and identification system 100. The system 100 can include a device 102 having a platform 104 with aligned carbon nanotube (CN×CNT) arrays 106 decorated with gold (Au) nanoparticles. The arrays 106 can be configured to capture species 108 (e.g., viruses, particles, etc.) within inter-tubular distances (ITDs 110) of the nanotubes. The system 100 can further include a spectrometer 112 configured for optical identification of species 108 captured by the device 102. The system 100 can further include a computer system 114 comprising a computer device 116 and a database 118. The data collected by the spectrometer 112 can be transmitted to the computer system 114 for storage and analysis. Machine learning techniques can be utilized to facilitate quick and accurate identification of the captured species 108.

The device 102 includes a casing 120 configured to house a platform 104. The platform 104 includes a substrate with CN×CNT arrays 106 attached thereto. In some embodiments, the device 102 is configured to be a portable handheld device. The substrate can be flat, curved, undulated, etc. The substrate can be made from silicon, glass, quartz, sapphire, metal, polymer, silicon dioxide, optical fibers, alumina, boron nitride, silicon carbide, etc. The casing 120 can be configured to bond to the substrate and seal the CN×CNT arrays 106 therein. In some embodiments, the casing 120 can be configured to have a cover that is removably attached so as to grant access to the platform 104 housed therein. The casing 120 can be bonded to both the substrate and the nanotubes forming the CN×CNT arrays 106, bonded to just the substrate, boned to just the CN×CNT arrays 106, or any combination thereof. The casing 120 includes a top 122, a bottom 124, and sides 126. In some embodiments, the substrate and/or CN×CNT arrays 106 is/are only bonded to the top 122 or a portion of the top 122. In some embodiments, the substrate and/or CN×CNT arrays 106 is/are only bonded to the bottom 124 or a portion of the bottom 124. In some embodiments, the substrate and/or CN×CNT arrays 106 is/are bonded to the top 122 and the bottom 124 or a portion of the top 122 and the bottom 124.

The casing 120 can be made from plastic, metal, glass, sapphire, polymer, polydimethylsiloxane (PDMS), etc. The CN×CNT arrays 106 can be aligned, which can include being vertically aligned (e.g., aligned in an orientation with respect to the casing top 122 and casing bottom 124). The casing 120 can have at least one inlet 128 and at least one outlet 130. In operation, solution containing species 108 (e.g., particles, viruses, etc.) can be caused to enter the device 102 via the inlet 128, passes over or through the CN×CNT arrays 106, and exit via the outlet 130. As the solution passes through the device 102, at least some of the species 108 in the solution are captured by the ITDs 110 of the nanotubes based on the size of the species 108. The device 102 can be configured to process volumes of solutions within a range from microliters to liters.

The nanotubes of the CN×CNT arrays 106 can include a single-walled CNT, a double-walled CNT, a multi-walled CNT, or any combination thereof. The nanotubes of the CN×CNT arrays 106 can be doped. For instance, nanotubes of the CN×CNT arrays 106 can be nitrogen-doped, boron-doped, silicon-doped, aluminum-doped, phosphorus-doped, lithium-doped, or combination thereof. Any one or combination of nanotubes within the CN×CNT arrays 106 can be doped with the same or different dopant than another nanotube. The dopant can be selected to enhance biocompatibility for maintaining the viability of captured species 108. The ITDs 110 of the nanotubes can be within a range from 1 nm to 750 nm. Any one or combination of nanotubes within the CN×CNT arrays 106 can have an ITD 110 that is the same or different than that of another nanotube. In addition, the ITD 110 of a nanotube can vary along a length of the nanotube.

Constructing the platform 104 can involve a stamping process and a functionalization process.

The stamping process can involve patterning catalytic particles on the substrate. For instance, the method can involve preparing a mold to grow the nanotubes and to pattern catalytic particles on the substrate. The mold can be a micro-mold made (e.g., 3-D printing or other additive manufacturing) from a polymer or other suitable material. The mold can have a pattern to it. This can include a spiral pattern, a zig-zag pattern, a herringbone pattern, etc. This pattern can be 3-dimensional. The substrate (e.g., Si/SiO₂) can be introduced into the mold such that the pattern of the mold is imparted onto the substrate. The catalytic particles (e.g., particle precursors such as Fe for example) can be spin-coated onto the substrate. For instance, a solution of iron (III) nitrate nonahydrate, Fe(NO₃)₃.9H₂O can be spin-coated onto the substrate so that Fe precursors are patterned onto the substrate to form Fe catalytic particles thereon. This process is referred to herein as forming stamped particle (e.g., Fe) precursor arrays.

In some embodiments, the spin-coating can involve spin-coating different solutions, each solution having a different concentration of particle precursors. The stamped particle precursor arrays can then be used as catalytic substrates to grow arrays 106 of aligned CN×CNTs. For instance, chemical vapor deposition can be used to selectively grow CN×CNTs on the precursor particles to form the aligned nanotube arrays. The nanotubes can be porous or semi-porous. In some embodiments, the CN×CNTs can be grown via chemical vapor deposition while in the presence of benzylamine. The use of Fe(NO₃)₃.9H₂O can result in CN×CNT arrays 106 containing nitrogen dopants (N-dopants). Other precursor particles can be used, such as iron particles, iron oxide (Fe₃O₄) particles, compounds made of Fe, Co, Ni, oxides, nitrides, etc.

The stamping process disclosed herein allows for patterning of multiple zones of a 3-D patterned (e.g., herringbone) arrays 106 with different ITDs 110. For instance, the spin-coating of different concentrations of precursor particles can facilitate the formation of nanotube CN×CNTs having different ITDs 110. Different concentrations of solution precursors results in precursor particles of varying density and diameter, which lead to growing aligned CN×CNTs of different diameters and ITDs 110. The diameters of nanotubes and the ITDs 110 are a function of the concentration of the solution precursors, and thus the diameters of nanotubes and ITDs 110 of the CN×CNT arrays 106 can be tunable based on the concentrations of the different solution precursors used. It should be noted that the density of the CN×CNT arrays 106 are dependent on the diameters of nanotubes and the ITDs 110, and thus is also dependent on the concentration of the solution precursors. The pattern of the nanotubes imposed by the mold (e.g., the herringbone pattern), the different nanotube diameters, and the different ITDs 110 allow for capturing of species 108 of different sizes.

The functionalizing process can involve coating the nanotubes of the CN×CNTs arrays 106 with species 108 enriching particles. The enriching particles can range in size from 1 nm to 30 nm. The enriching particles can be configured to enhance the sensitivity that can be obtained from spectroscopy measurements. The sensitivity is enhanced due to enhancing the intensities obtained from spectrometry. For instance, the nanotubes of the CN×CNTs arrays can be coated with Au nanoparticles to generate Au/CN×CNTs arrays. Other enriching particles can be used, such as chemicals, molecules, antibodies, etc. These can include gold particles, silver particles, etc. In addition, nanoparticles of different shapes and sizes (e.g., from 1-100 nm) can be used. The 3-D patterned nanotubes of the enriched-CN×CNTs arrays 106 enhance interactions between the species 108 and the enriched-CN×CNT arrays 106 through induced chaotic flow—e.g., enriched-CN×CNT arrays 106 enrich species 108 by removing host contaminates (e.g. debris, cells, free-floating nucleic acids, etc.). The enrichment of the species 108, via functionalized CN×CNTs, allows for optical detection of species 108 captured by the device 102. For instance, surface-enhanced Raman spectroscopy can be used to detect species 108 captured by the device 102. It should be noted that captured species 108 can be detected via spectroscopy in a label-free manner (e.g., in a manner that obviates use of an antibody or a primer as a label); however, labeling techniques can be used to augment the detection.

It is contemplated for the spectrometer 112 to be a Raman spectrometer, but other spectrometers can be used (e.g., infrared spectroscopy, fluorescent spectroscopy, Fourier-transform infrared spectroscopy, second harmonic spectroscopy, etc.). The spectrometer 112 can be used to perform in-situ Raman spectroscopy to optically detect and capture species 108 via optical spectroscopic techniques configured to identify the chemical structure of the species 108 by providing its structural/vibrational fingerprint. With conventional methods, the efficiency of the Raman scattering signal can be very weak—approximately 1 out of 10⁶ phonons is absorbed and emitted through Raman (inelastic) scattering. This weak efficiency significantly limits the signal intensity of Raman spectra, thus constraining its application. However, functionalization via enriching particles (e.g., Au nanoparticles) can enhance localized plasmon resonance (LSPR), which can enhance the signal of Raman scattering up to ˜108. This Raman signal enhancement can facilitate detection of species 108 down to a single molecule level (˜picomolar). For instance, embodiments of the system 100 can be used to identify compositions and quantities of different surface molecules, including any chemical compounds such as proteins, amino acids, nucleic acids, etc.

The computer system 114 includes a computer device 116 and a database 118. The computer system 114 is in operative association with the spectrometer 112 so as to receive spectrometer data from the spectrometer 112. Using machine learning techniques, the computer system 114 compares fingerprints of the spectrometer spectra to identify species 108 captured by the device 102. With machine learning, the system 100 can identify species 108 within several minutes and without any bench top procedures or requiring any labeling techniques. When the computer device 116 receives spectra data of from the device 102, the computer device 116 acquisitions data from the database 118 regarding historical spectra data of different species 108 (e.g., >1000 spectra of different species) anticipated to be captured for a particular application and makes a comparison to the spectra data being obtained from the spectrometer 112 to identify the species 108 captured. Machine learning is used to assist with the comparison. For instance, the inventors discovered that with species 108 that are viruses, different strains cluster separately. Thus, a machine learning-based strategy to identify different virus strains included a 3-fold cross-validation to determine the best classification model out of four candidates: logistic regression, support vector machine, decision tree, and random forest. The logistic regression model minimized multinomial loss, thereby achieving the highest validation accuracy. Thus, while any one or combination of the four disclosed machine learning techniques above can be used, logistic regression may be preferred for species 108 that are viruses.

Embodiments of the computer device 116 can be a processor in operative association with a memory. The processor can be one of a scalable processor, parallelizable processor, and optimized for multi-thread processing capabilities. In some embodiments, the computer device 116 can be a supercomputer or a quantum computer whose processing power is selected as a function of anticipated network traffic (e.g. data flow). The processor can include any integrated circuit or other electronic device (or collection of devices) capable of performing an operation on at least one instruction including, without limitation, a Reduced Instruction Set Core (RISC) processor, a CISC microprocessor, a Microcontroller Unit (MCU), a CISC-based Central Processing Unit (CPU), a Digital Signal Processor (DSP), etc. The hardware of such devices may be integrated onto a single substrate (e.g., silicon “die”), or distributed among two or more substrates. Various functional aspects of the processor may be implemented solely as software or firmware associated with the processor.

The memory can be optionally associated with the processor. Embodiments of the memory can include a volatile memory store (such as RAM), non-volatile memory store (such as ROM, flash memory, etc.), or some combination of the two. For instance, the memory can include, but is not limited to, RAM, ROM, EEPROM, flash memory or other memory technology CDROM, digital versatile disks (DVD) or other optical storage, magnetic cassettes, magnetic tape, magnetic disk storage or other magnetic storage devices, or any other medium which can be used to store the desired information and which can accessed by the processor. In some embodiments, the memory can be a non-transitory computer-readable medium. The term “computer-readable medium” (or “machine-readable medium”) as used herein is an extensible term that refers to any medium or any memory, that participates in providing instructions to the processor for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, and transmission media.

Transmission media includes coaxial cables, copper wire and fiber optics, including the wires that include or form a bus. Transmission media can also take the form of acoustic or light waves, such as those generated during radio-wave and infrared data communications, or other form of propagated signals (e.g., carrier waves, infrared signals, digital signals, etc.). Forms of computer-readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Instructions for implementation of any of the aforementioned methods can be stored on the memory in the form of computer program code. The computer program code can include program logic, control logic, or other algorithms that may or may not be based on artificial intelligence (e.g., machine learning techniques, artificial neural network techniques, etc.).

In some embodiments, the computer device 116 can be part of or in connection with a communications network. For example, the computer device 116 can include switches, transmitters, transceivers, routers, gateways, etc. to facilitate communications via a communication protocol that facilitates controlled and coordinated signal transmission and processing. The communication links can be established by communication protocols that allow computer device 116 to form a communication interface. The communication interface can be configured to allow the computer device 116 and another device (e.g., a computer device or processor) to form a communications network. The communications network can be configured as a long range wired or a wireless network, such as an Ethernet, telephone, Wi-Fi, Bluetooth, wireless protocol, cellular, satellite network, cloud computing network, etc. Embodiments of the communications network can be configured as a predetermined network topology. This can include a mesh network topology, a point-to-point network topology, a ring (or peer-to-peer) network topology, a star (point-to-multiple) network topology, or any combination thereof.

Embodiments of the system 100 can be used to capture different species 108 based on their size, enrich the species 108, and optically identify species 108. For applications in which the species 108 are unknown strains of viruses, identification can be achieved without extensive sample processing. It should be noted that, the viruses are still viable and can be further tested using conventional methods, such as electron microscopy, immunostaining, cell culture, next-generation sequencing, etc. even after capture and identification.

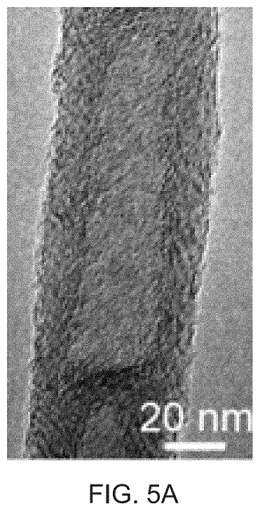

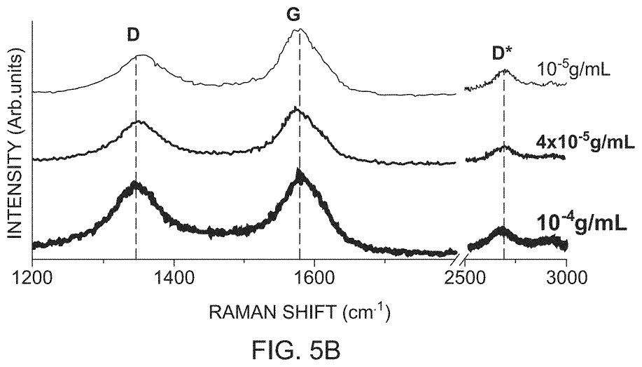

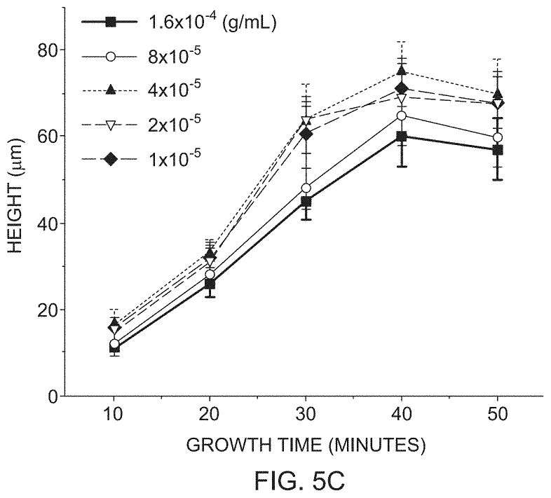

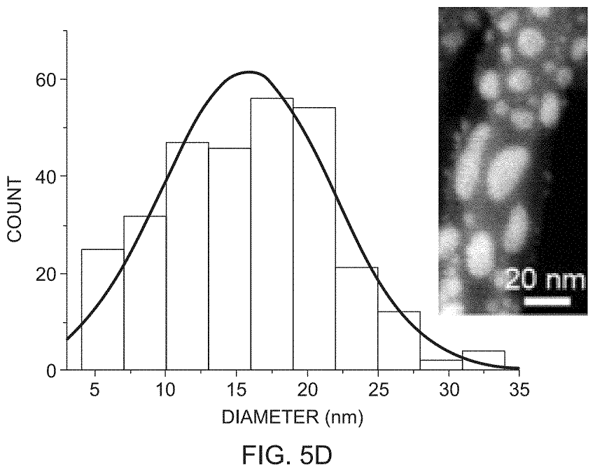

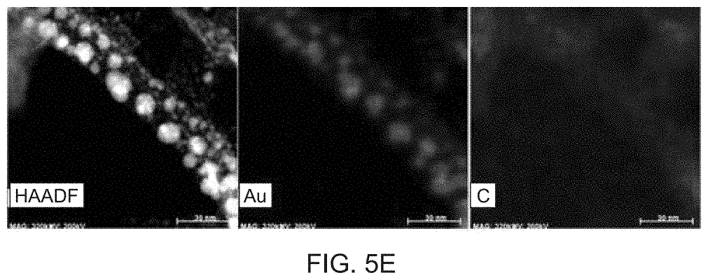

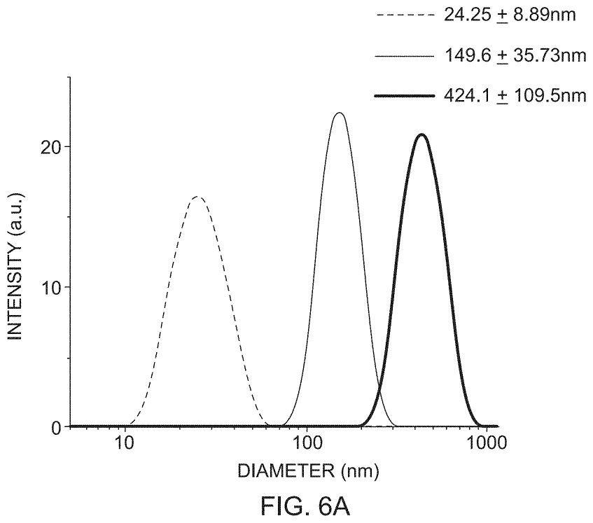

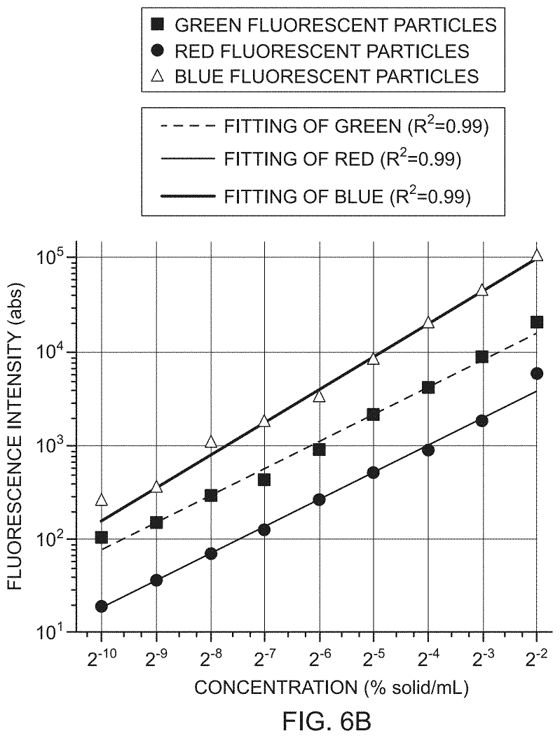

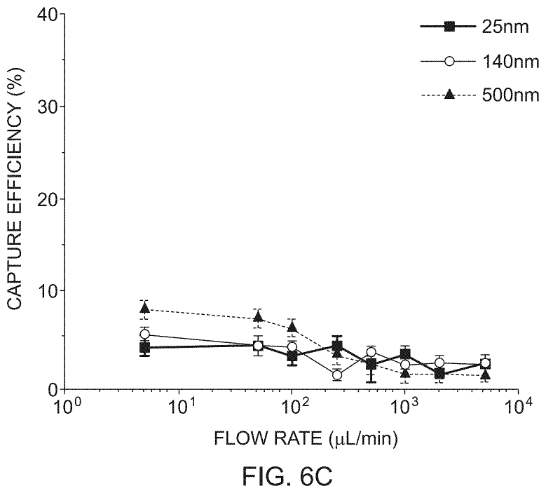

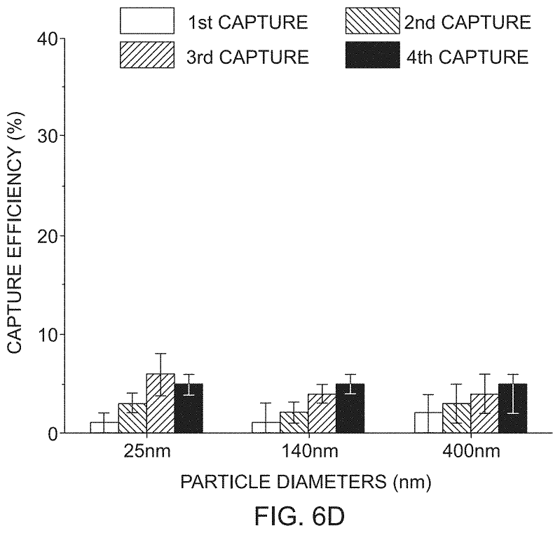

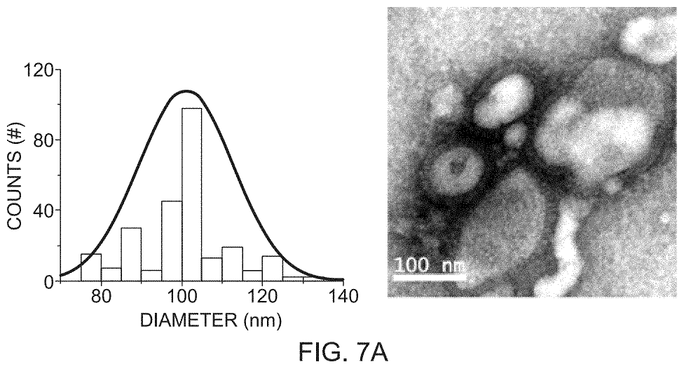

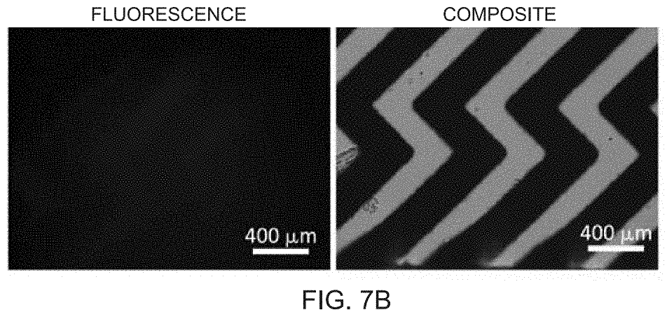

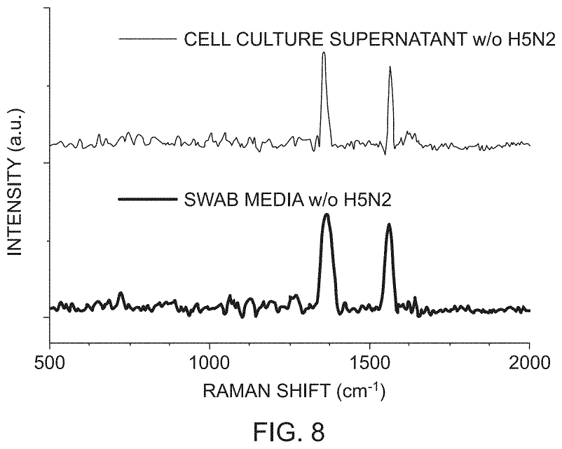

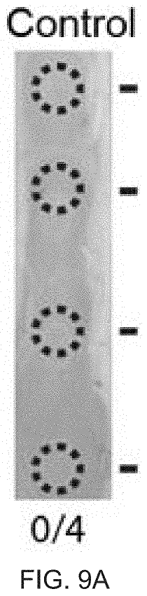

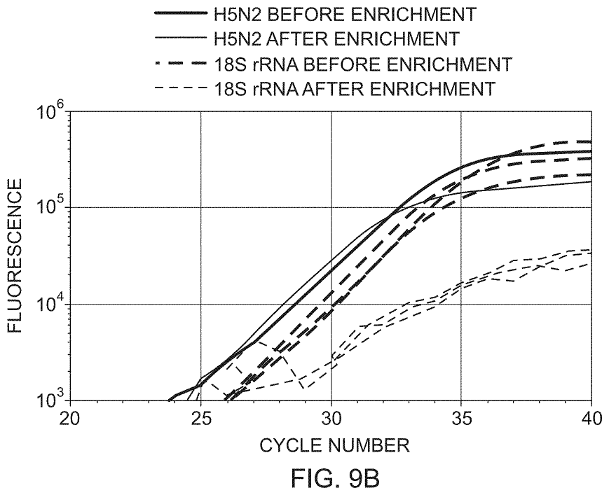

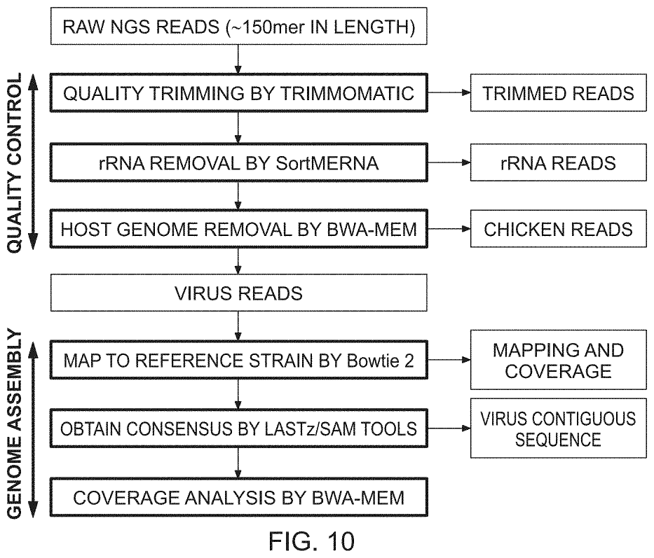

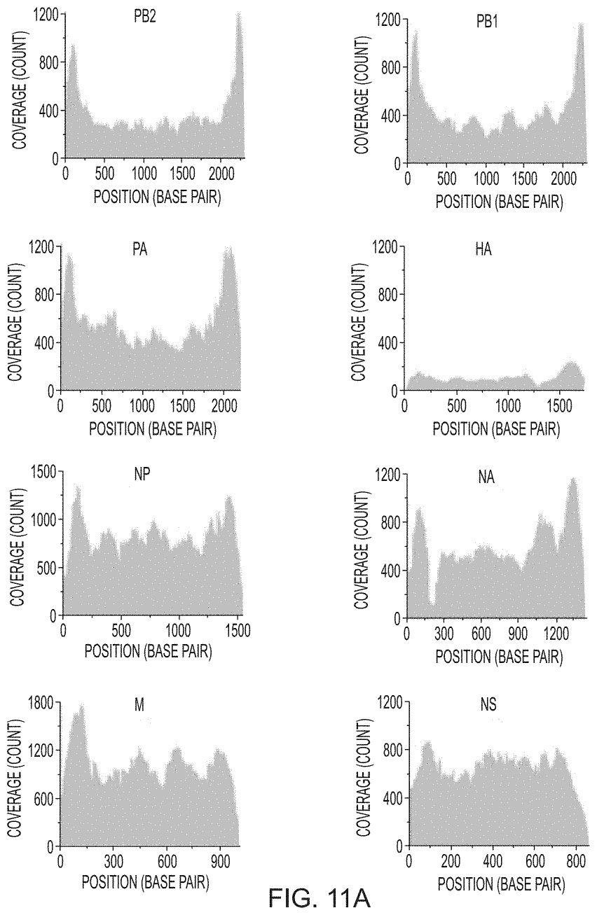

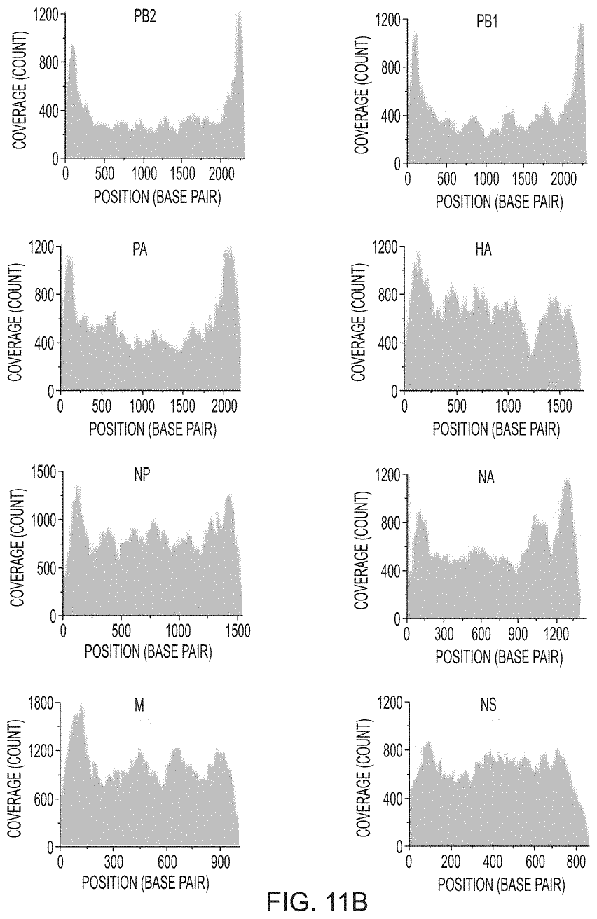

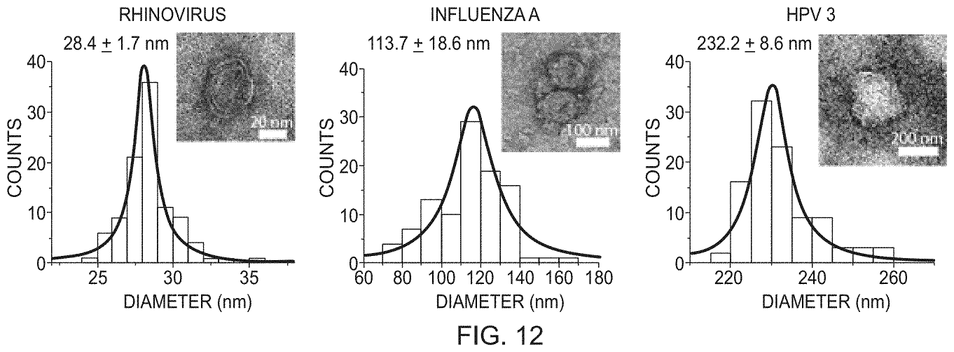

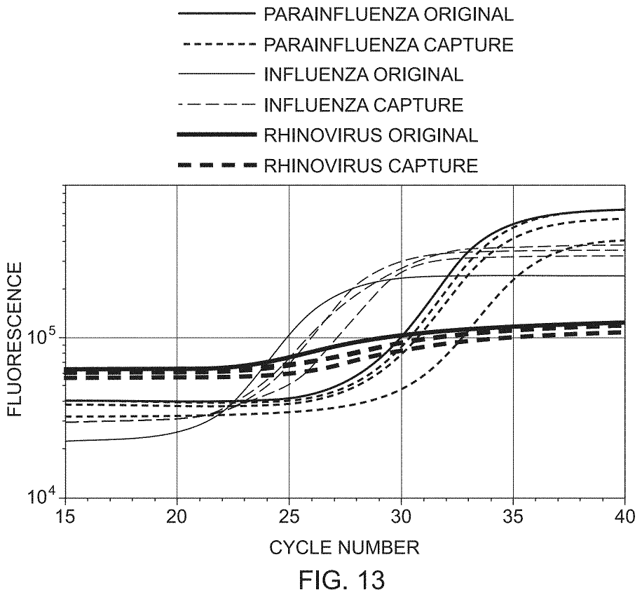

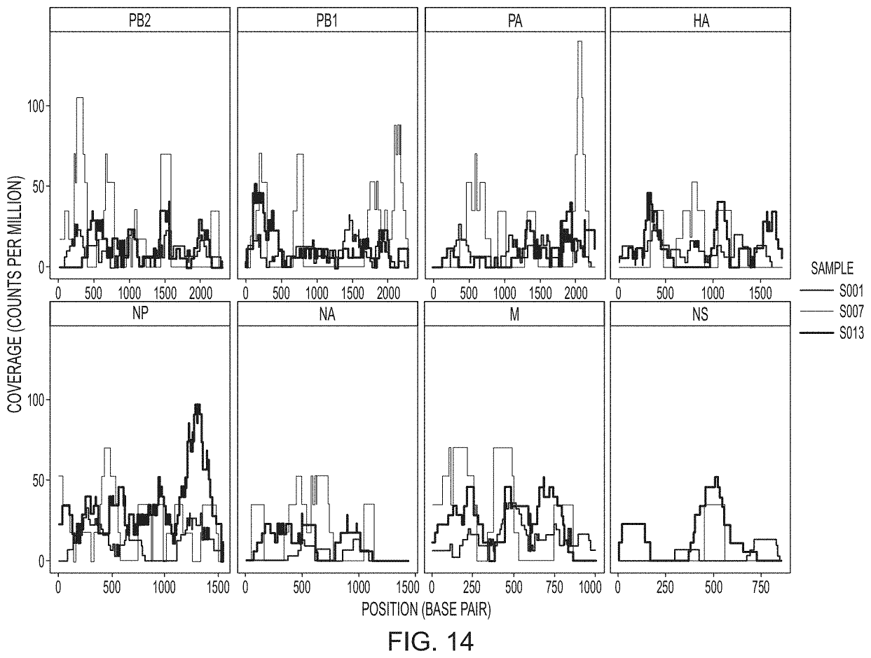

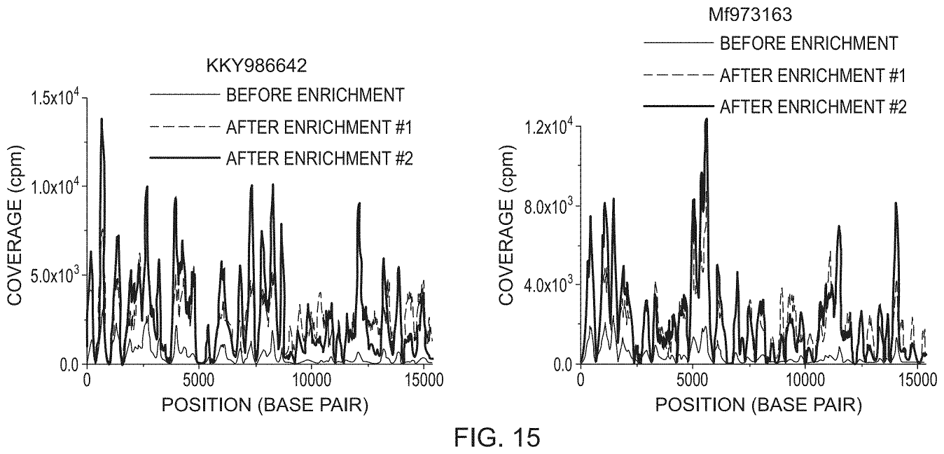

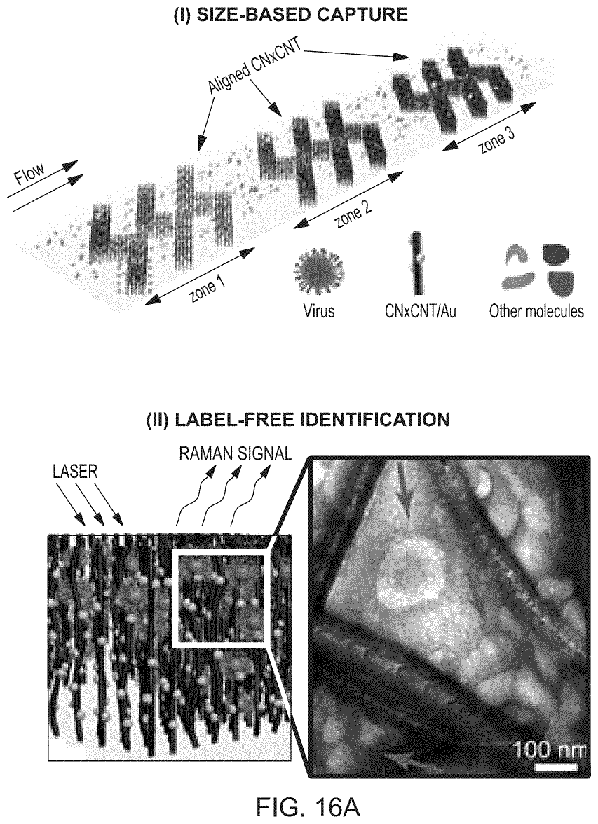

Referring to FIGS. 5-14, as noted herein, embodiments of the device 102 can be used to capture species 108 for analysis, wherein the ITDs 110 of the nanotubes can be tuned to a specific size for the purpose of capturing species 108 of a predetermined size. For instance, if a species 108 has a known size range, the ITDs 110 of the nanotubes can be tuned to capture species 108 of within that size range. Passing a solution containing the species 108 through the device 102 will enable users to determine whether the solution contains species 108 within that size range, due to those species being captured. The species 108 can then be analyzed via spectroscopy to identify the species 108. The identification of the species 108 can be used for diagnosis, quantification, etc. In an exemplary implementation, a solution containing the species 108 can be patient-derived solutions, including, but not limited to, blood, urine, saliva, tissue, stool, sweat, etc. The solution can be a gas, such as a patient's breath, wherein the species 108 may be air-borne pathogens. In some embodiments, the solution containing the species 108 can be obtained from non-human sources, such as animals, plants, insects, etc. In some embodiments, the solution can be air, sea water, etc.

Embodiments of the device 102 can allow for capture and release species 108 while maintaining the viability of the species 108. For instance, the species 108 can include, but are not limited to, viruses (such as plant viruses, human viruses, herpes, zika, hepatitis C, ebola), microorganisms and parasites (such as bacteria, amoeba, and plasmodium), and their various life stages, etc. The ITDs 110 of the nanotubes can be tuned to a specific size for the purpose of capturing such species 108. As noted above, the dopant can be selected to enhance biocompatibility for maintaining the viability of captured species 108. After a solution containing such species 108 is passed through the device 102, the removable portion of the casing 120 can be removed. At least a portion of the captured species 108 can be released. The species 108 can be released by scratching the platform 104 surface, degrading the platform 104 nanostructure, etc. Once released, the species are still viable and can be subjected to further testing (e.g., as electron microscopy, immunostaining, cell culture, next-generation sequencing, etc.).

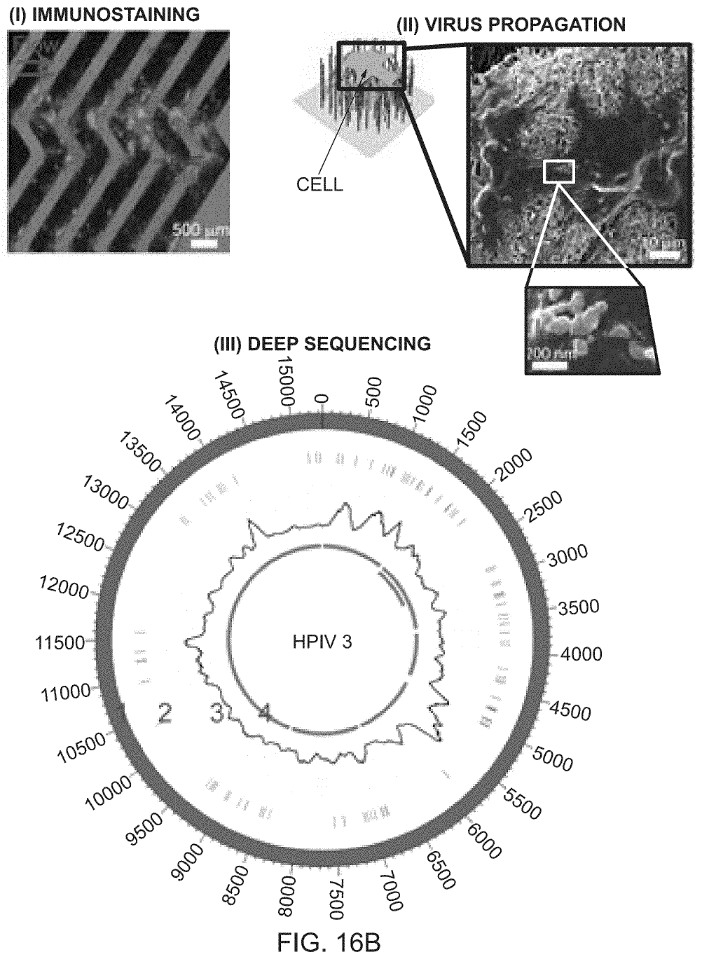

Experimental tests were conducted to assess the effectiveness and efficiency of the system 100. The results are presented below. In the below discussion, the device may be referred to as the VIrus capture with Rapid Raman spectroscopy detection and IdentificatiON (VARRION) platform. The VIRRION platform was constructed with aligned nitrogen-doped carbon nanotube (CN×CNT) arrays, decorated with gold (Au) nanoparticles (See FIG. 16). The inventors discovered that the CN×CNT had better biocompatibility than its pristine form. For VIRRION, CN×CNTs were used as the building blocks but a stamping technique was added to pattern Fe catalytic particles. After chemical vapor deposition (CVD), CN×CNTs grew on Fe particles and formed aligned nanotube arrays. The nanotube arrays were then coated with Au nanoparticles (Au/CN×CNTs) for obtaining enhanced Raman spectroscopy intensities. This stamping route allowed us to pattern multiple zones of the herringbone arrays with different inter-tubular distances (ITDs), ranging from 22±5 nm to 720±64 nm, to match a variety of viruses of different size. Studies have shown that herringbone patterns, made of PDMS (polydimethylsiloxane), enhance mixing of the samples inside a microfluidic channel by inducing chaotic flow. We adopted the herringbone design but constructed them by using the aligned Au/CN×CNTs. This 3-dimensional and porous herringbone array could enhance interactions between viruses to the Au/CN×CNT arrays through induced chaotic flow.

During capture, our data showed that Au/CN×CNT arrays enriched virus particles by removing host contaminates (e.g. debris, cells, and free-floating nucleic acids). In order to optically detect the captured viruses, we integrated Raman spectroscopy in conjunction with a machine learning algorithm. By comparing fingerprints of the virus spectra, this approach identified different viruses within several minutes without any bench top procedures such as pipetting nor requiring any label (e.g. antibody or primer, etc.). More importantly, we noted that after capture and detection, the viruses were still viable and could be further tested using conventional methods, such as electron microscopy, immunostaining, cell culture, and next-generation sequencing. Our results also showed that this enrichment promoted these conventional methods by preparing virus sample in an effective way.

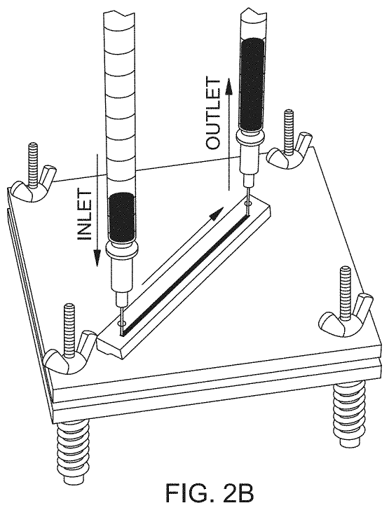

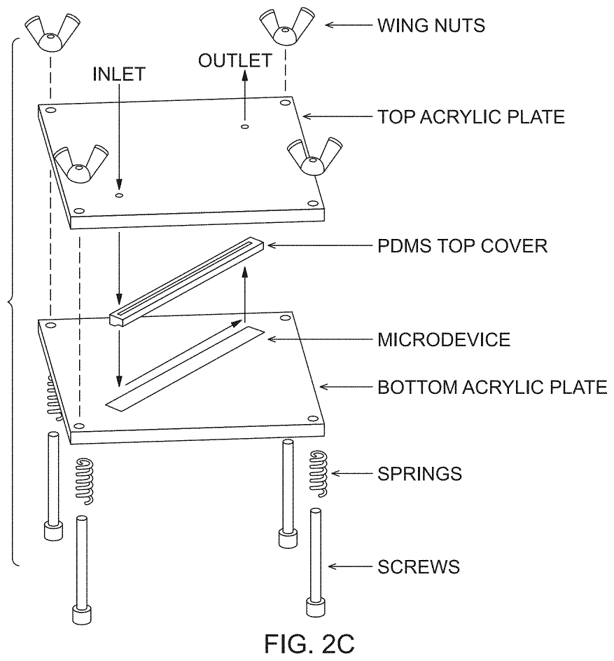

We expanded on the process of applying an expensive and laborious micro-lithography-based technique to selectively grow aligned CN×CNT arrays on silicon substrates by developing a stamping technique to easily pattern catalytic Fe particles used to grow hearing-bone arrays of aligned CN×CNT with a gradient of ITDs that match the sizes of different viruses (See FIG. 2). We used a commercial 3D printer to prepare polymer micro-molds exhibiting herringbone micro-patterns that were used to deposit different concentration solutions of Fe precursors onto Si/SiO₂ substrates, and to grow aligned CN×CNTs with tuned ITDs. The stamping technique started by spin-coating iron (III) nitrate nonahydrate, Fe(NO₃)₃.9H₂O, on Si/SiO₂ substrates. Subsequently, each 3D-printed mold patterned different concentrations of the Fe-based precursors to the substrate by stamping. These stamped precursor arrays were then used as catalytic substrates to grow arrays of aligned CN×CNTs using chemical vapor deposition (CVD) in the presence of benzylamine. The height of the aligned CN×CNTs reached ˜70 μm after growing CNTs for 40 minutes. Structural characterization of the grown nanotubes revealed compartmentalized bamboo-like tubular structures containing N-dopants, as observed by high resolution transmission electron microscopy (HRTEM) and Raman spectroscopy. Thus, our aligned carbon nanotubes contained nitrogen dopants and were successfully synthesized by the stamping technique.

During CN×CNT CVD growth, we observe how Fe particles form upon heating the Fe based precursors (Fe(NO₃)₃.9H₂O). These newly formed Fe particles act as catalytic nucleation sites responsible for growing aligned CN×CNTs. We observed that different concentrations of Fe-based precursors resulted in Fe particles of varying density and diameter that were responsible for growing aligned CN×CNTs of different diameters and ITDs. Our results indicate that by increasing the concentration of Fe-based precursor, both the diameter and density of Fe-rich particles and CN×CNTs also increased. More importantly, we determined that a CN×CNT array has tunable ITDs that are a function of the concentration of Fe-based precursors, and ranged from 22±5 nm to 720±64 nm. This stamping technique allows patterning a gradient of aligned CN×CNT arrays with controlled dimensions in a simple and cost-effective way. Furthermore, the tunable ITD covers the range of most virus sizes.

In order to optically detect the captured viruses using a label-free approach, we functionalized CN×CNTs with Au nanoparticles to enable surface-enhanced Raman spectroscopy (SERS). Raman spectroscopy is an optical spectroscopic technique that is commonly used to identify the chemical structure of substances by providing its structural/vibrational fingerprints. However, the efficiency of the Raman scattering signal is usually very weak—approximately 1 out of 10⁶ phonons is absorbed and emitted through Raman (inelastic) scattering. This weak efficiency dramatically limits the signal intensity of Raman spectra, thus constraining its application. However, studies have shown that Au nanoparticles enhance localized plasmon resonance (LSPR), which can further enhance the signal of Raman scattering up to ˜10⁸. Interestingly, this Raman signal enhancement enables the detection down to a single molecule level (˜picomolar).

We observed that Au nanoparticles of ˜15 nm in diameter are well-distributed on CN×CNT. Results of the UV-Vis measurements indicate that the Au/CN×CNT arrays have a strong localized surface plasmon resonance. We characterized the sensitivity and uniformity of the SERS detection using a reference Raman dye, Rhodamine 6G (R6G). In brief, the characterization shows that the Au/CN×CNTs arrays provide uniform, consistent, and sensitive (10⁻¹⁰M) SERS signals across different ITDs.

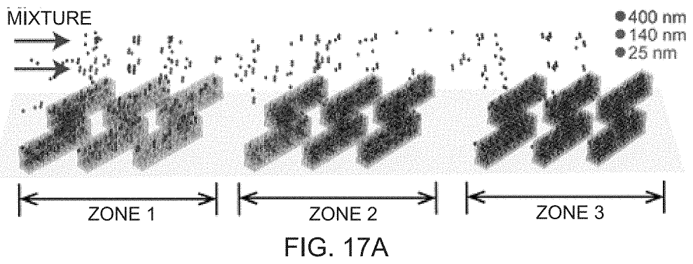

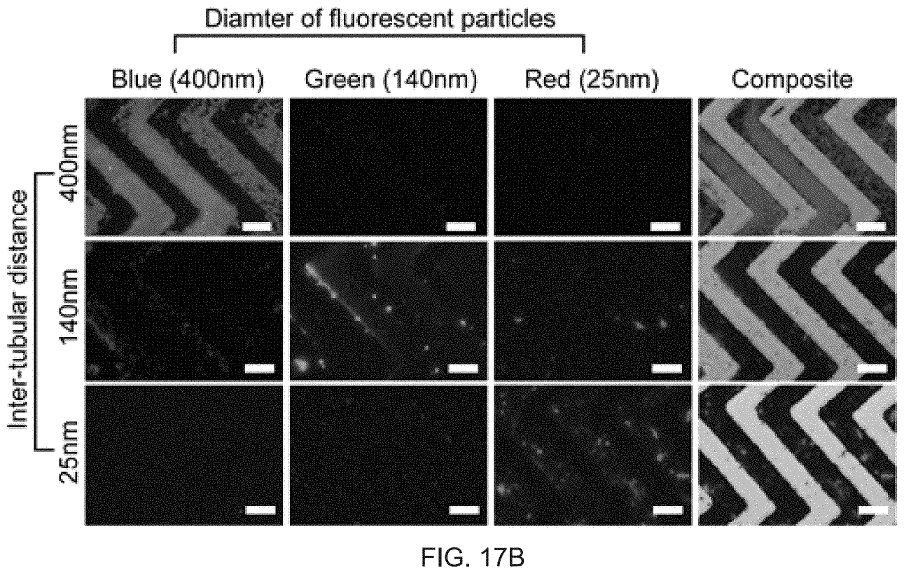

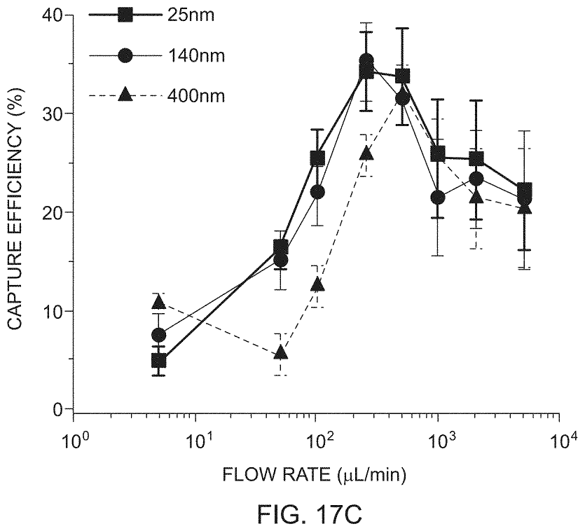

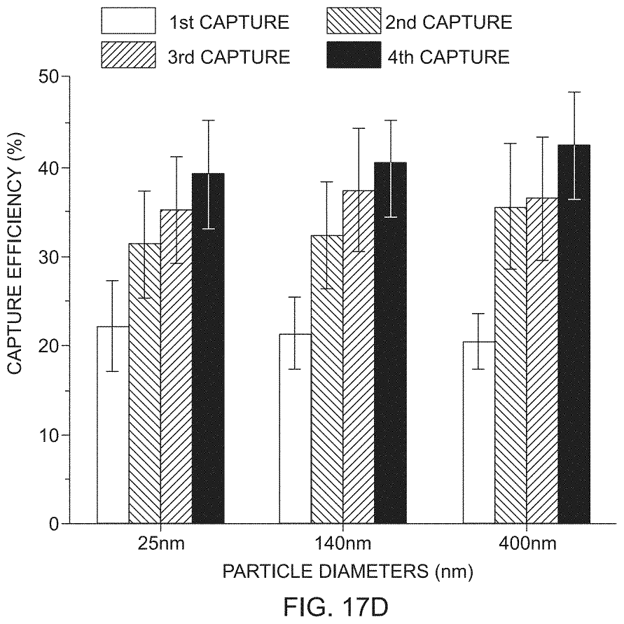

After growing the CN×CNT arrays and fabricating micro-fluidic devices, we characterized the range of the size-based capture by using different sizes of fluorescently-labeled silica particles. (See FIG. 17). We mixed together fluorescently-labeled silica particles of different diameters (400 nm, 140 nm, and 25 nm) and of different fluorescent wavelengths (415 nm, 508 nm, 612 nm, respectively. To capture and separate each subgroup of silica particles in the mixture, we assembled a microdevice with three zones of aligned CN×CNT arrays exhibiting 400, 140, and 25 nm ITDs to match their sizes. To characterize the efficiency of the size-based capture, we calculated capture efficiencies under different flow rates by measuring fluorescent intensities of the flow through and dividing by the intensities of the originals. After capture, strong fluorescent signals of individual subgroups of silica particles were detected in the CN×CNT array zones, where ITDs matched the silica bead sizes. Under fluorescent microscopy, it is clear that VIRRION successfully captured and separated different sizes of particles from the mixture. When the flow rate ranged between 250 μL/min and 500 μL/min, capture efficiency reached 31.8±3.3%, 35.3±4.7%, and 34.3±4.5%, respectively for silica particles of 400, 140, or 25 nm in diameter. Under these flow conditions, the herringbone structured CN×CNT arrays enhanced mixing of the sample during transport through a microfluidic channel, which increased the interactions between silica particles and CN×CNT arrays. Notably, under flow rates that mimicked a manual push through a syringe, 4×10³ μL/min, the capture efficiencies could still reach ˜22%. To confirm the size-based capture contributed by the CN×CNT arrays, we assembled micro-devices without CN×CNT arrays, as a control experiment. After passing through the same mixture of the particles, very few particles (˜4%) were captured. The results affirmed that the CN×CNT arrays effectively captured different sizes of the particles.

We repeated the capture by loading the microfluidic device flow-through back into the same VIRRION through a manual push through a syringe to test whether the capture efficiency could be improved. The results indicated that the capture efficiency increased by ˜10% after 2^{nd} capture. After 4th capture, the efficiency increased another 10% on average that combined efficiencies became 42.4±6.3%, 40.5±5.4%, and 39.2±6.1% for the 400, 140, 188 and 25 nm silica particles, respectively. Meanwhile, micro-devices without CN×CNT arrays, the control experiment, showed low capture efficiencies (˜6%). These results demonstrate that the VIRRION can selectively capture different sizes of silica particles from a mixture, even at low flow-through pressure when using a syringe.

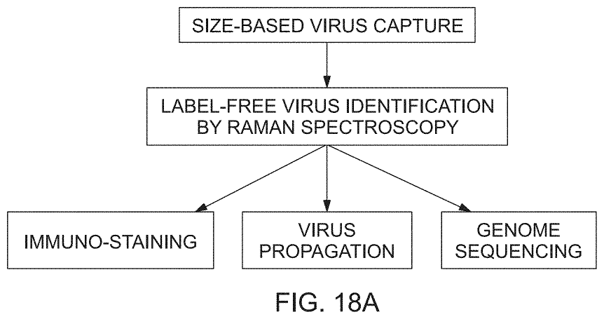

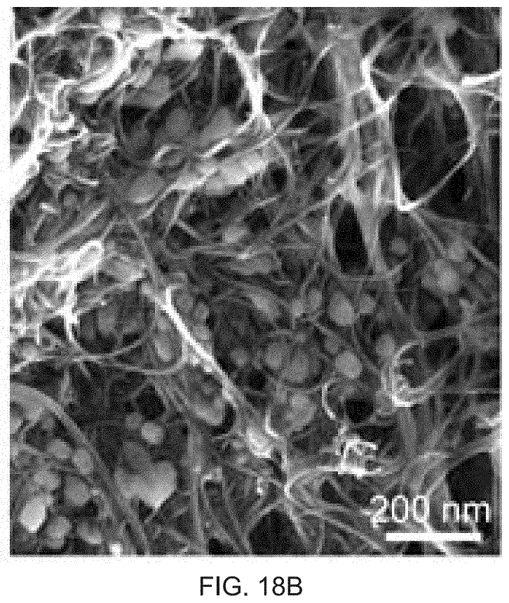

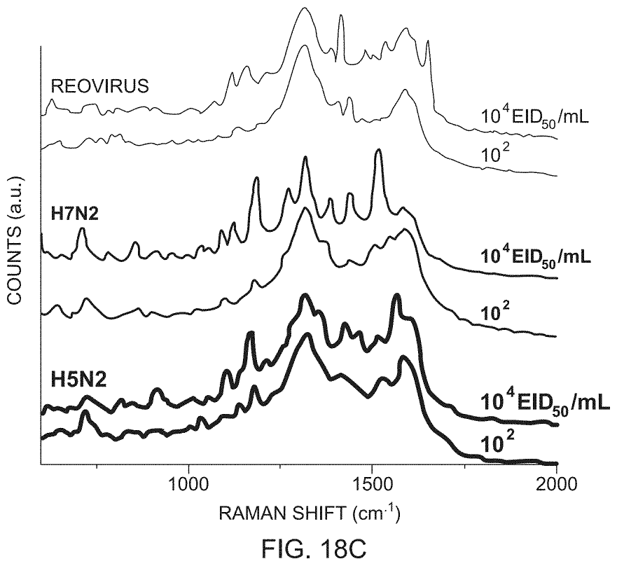

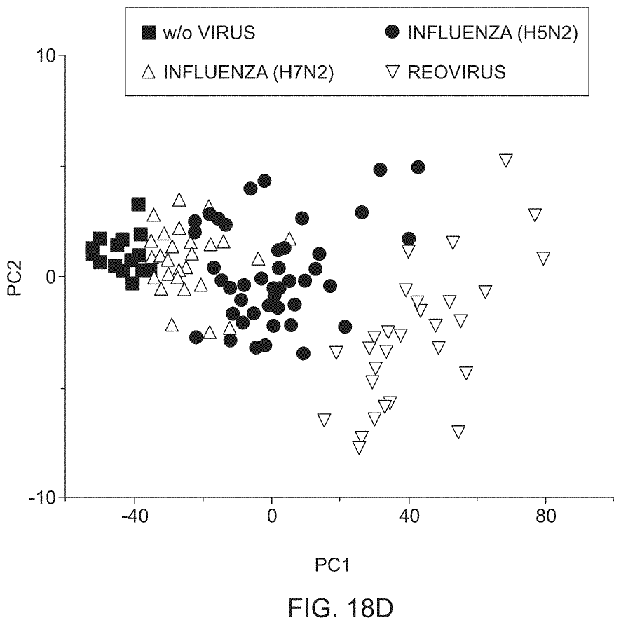

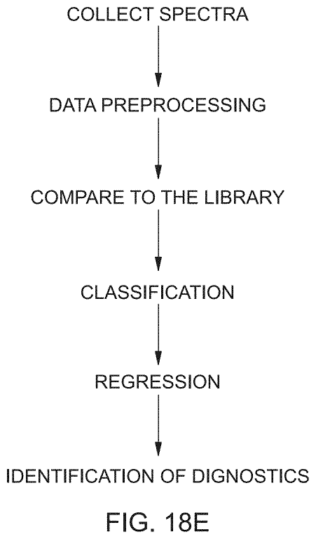

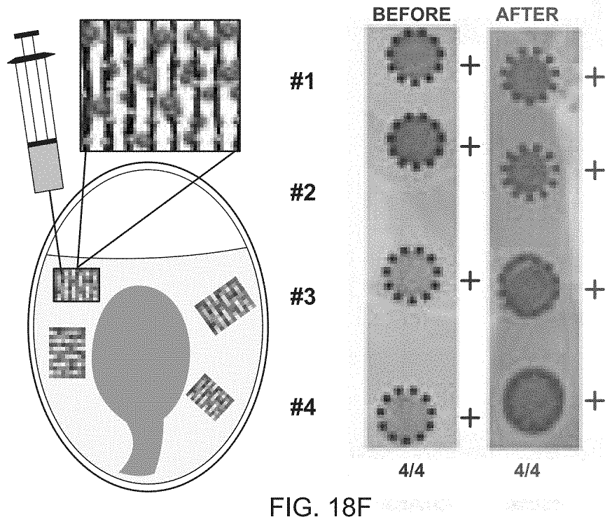

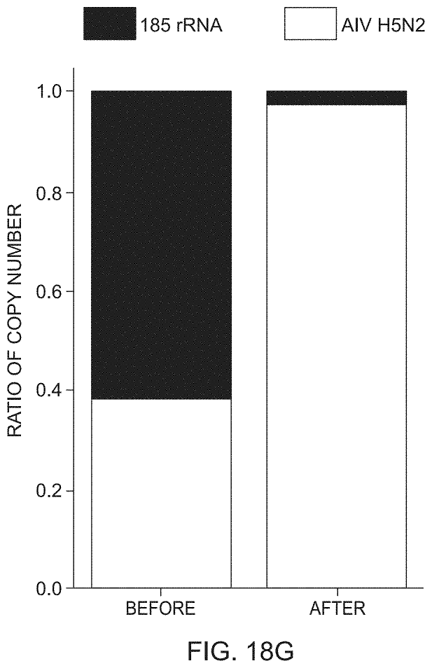

RNA viruses evolve rapidly and are the main viral agents that cause emerging infections. We used a low pathogenic avian influenza virus (LPAIV), an RNA virus, to test the efficacy of the VIRRION platform (See FIG. 18). The average size of the H5N2 LPAIV is 101±11.7 nm, measured by transmission electron microscopy (TEM). We assembled a VIRRION with an ITD of 100 nm to match the average size of the AIV. We manually pushed through a syringe into a VIRRION 5 mL of the virus sample containing 10⁴ EID₅₀/mL H5N2. We then performed an immunofluorescence assay on the VIRRION to detect captured AIV in-situ by using an AIV H5 subtype-specific monoclonal antibody. Strong fluorescent signals were detected on the CN×CNT arrays while no fluorescence was detected on the VIRRION when processing control samples that did not have H5N2. Under scanning electron microscopy (SEM) and TEM, we clearly observed virus particles captured by the VIRRION.

To perform fast and in situ optical identification of the virus particles captured within the nanotube array via Raman spectroscopy, we developed an algorithm using a spectra database of three different viruses: strains from two AIV subtypes (H5N2 and H7N2) and Reovirus. We first collected multiple Raman spectra after VIRRION capture of these viruses to build a simple database. Surface enhancement of the Raman scattering happens at a so-called ‘hot spot’, i.e. the 1 nanometer region between two adjacent gold nanoparticles. Thus, when viruses are trapped between the Au/CN×CNT arrays, the only Raman signal from the virus, in the vicinity of the hot spot, is enhanced. We recorded Raman signals and averaged over 100 spectra for each strain to generate a reliable average fingerprint for each virus. Each virus strain has a distinct fingerprint that was still distinguishable at concentrations as low as ˜102 EID₅₀/mL. It is noteworthy that this sensitivity is equivalent to that of RT-qPCR detection. In addition, no prominent peaks were detected in the negative controls. We the applied a principal component analysis (PCA) to classify all viral Raman spectra. Our results indicate that different strains cluster separately. To develop a machine learning-based strategy to identify different virus strains, we used a 3-fold cross-validation to determine the best classification model out of four (logistic regression, support vector machine, decision tree, and random forest). The logistic regression model minimizing multinomial loss achieved the highest validation accuracy (˜74%) in differentiating between H5N2, H7N2, Reovirus, and the negative control.

We noticed that since H7N2 and H5N2 shared similar spectra, as expected, this resulted into large overlapping regions after classification, which challenged accuracy in distinguishing them apart. After capturing and identifying H5N2 virus particles, we performed cell culture to propagate captured H5N2 on the VIRRIONs. Notably, during culture the host cells attached and proliferated directly on the Au/CN×CNT arrays. The virus-like particles were observed on the surface of the cells, indicating effective replication and production of virus progeny. To confirm results of virus replication, we collected the supernatant and determined it contained a virus titer of 10⁶ EID₅₀/mL—similar to control experiments in which the virus is propagated in the same cell line in a culture flask. We obtained a higher virus titer, ˜10⁷ EID₅₀/mL, when we propagated captured viruses using embryonated chicken eggs (ECE). To do that, we disassembled the VIRRION, collected the Au/CN×CNT with embedded viruses, and directly inoculated these into ECE for AIV propagation. For both cell culture and ECE propagation, we also confirmed viral replication by Dot-ELISA28. These results clearly show that VIRRION maintains viability of the viruses during capture, and that it is possible to apply conventional isolation approaches directly on-chip.

We further analyzed the captured viruses by next-generation sequencing (NGS) to characterize the captured virus population without prior knowledge of the strains. To demonstrate the efficiency of enrichment of the VIRRION array and the reduction of host nucleic acid, we targeted the 18s rRNA, an essential housekeeping gene that is conserved in all eukaryotic cells. We used H5N2 spiked-in samples and extracted total RNA from the VIRRION after capture. We then characterized the enrichment by measuring the ratio of the copy number of host 18S RNA to virus hemagglutinin (HA) gene of H5N2 by RT-qPCR. Before enrichment, the original ratio of 18S rRNA to H5N2 viral RNA was 1:0.61. After enrichment, the ratio was 1:42.47 (18S rRNA to H5N2), thus illustrating that H5N2 is enriched ˜69. To further test enrichment by NGS, we captured a mixture of samples containing more than one strain of viruses since samples can be carrying more than one virus type or strain. When sequencing this type of sample, it is difficult to enrich without introducing bias. We prepared samples by spiking equal volumes of H5N2 and H7N2 with a HA titer of 2⁹ measured by hemagglutination inhibition assay ₂₉, into viral transport media (BD, #220531), and running the pooled viruses on a VIRRION using a 100 nm ITD. After enrichment, eight complete genomic segments for each strain were sequenced. We compared viral reads of the HA gene before and after enrichment. Interestingly, before enrichment, the H5N2:H7N2 ratio based on HA viral reads was 1:7.8, where 344 reads were from H5N2 and 2,693 reads from H7N2. After enrichment, the ratio was relatively well-preserved at 1:6.5, but with an increase in viral reads (1,314 reads for H5N2 and 8,568 reads for H7N2). There appeared to be a more than 3-fold enrichment for both viruses. However, this difference in enrichment efficiencies maintained the stoichiometric proportion of viruses present in the original samples. Moreover, the complete genome of both strains was sequenced with an average coverage of 519X for H5N2 and 599X for H7N2. In addition, after enrichment, the host (chicken) reads decreased from 68.44% (reads) of the total reads (547,998 of 800,699 reads) to 34.2% (297,157 reads) of the total reads (866,855 265 reads). These results indicate that the VIRRION can capture different strains of viruses and minimize artifacts during enrichment while removing sequencing reads contributed from the host.

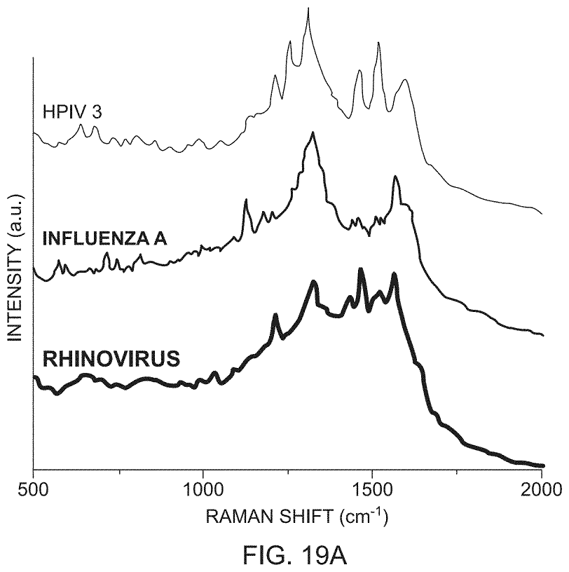

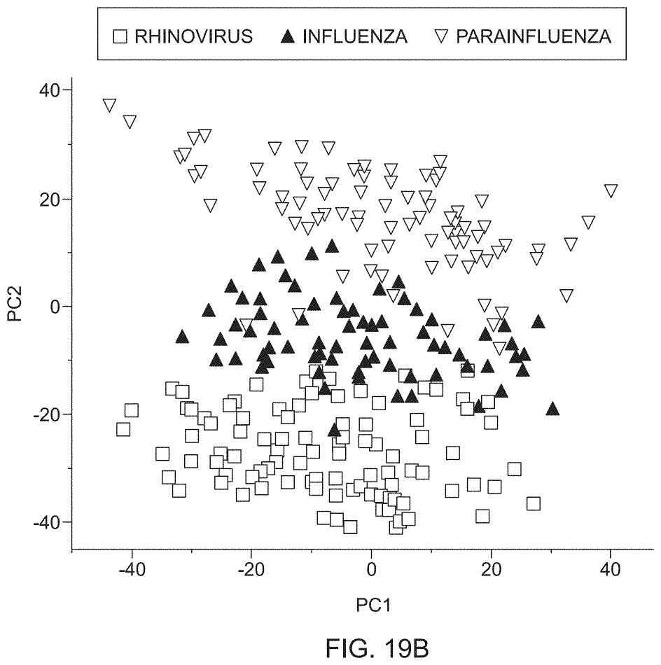

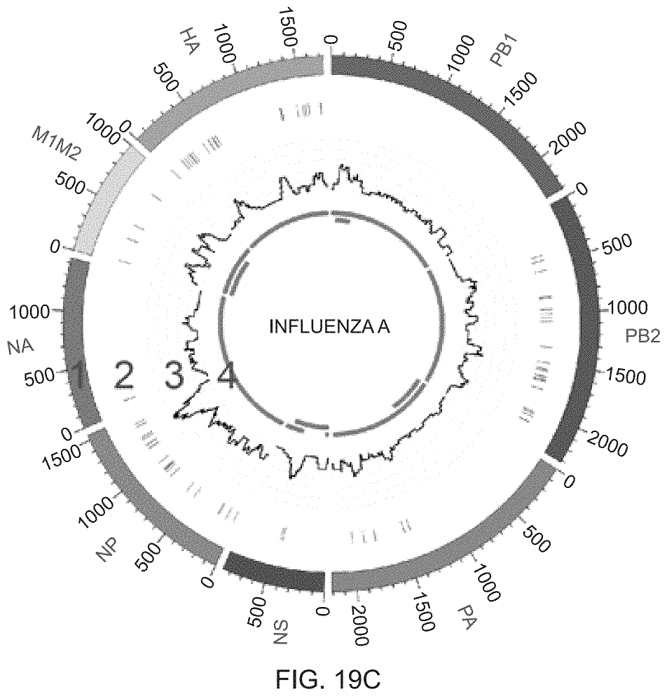

Acute respiratory infections are the third most common cause of death worldwide and responsible for 4 million deaths each year, which is 7% of all deaths annually. Clinical impact increases when they infect children, the elderly, and immunocompromised individuals. Most clinical or field samples have very low virus titers, and sequencing of total RNA usually results in a majority (>80% of the sequence reads) of host sequences, such as ribosomal RNA, rather than viral sequences. When developing methods for enrichment, the challenge is to minimize bias and to process clinically-relevant sample volumes. We validated the VIRRION in human respiratory infection diagnostics by rapidly capturing and identifying different viruses in respiratory samples from patients who had been diagnosed with rhinovirus, influenza virus type A (Influenza A), or Human parainfluenza virus type 3 (HPIV 3). Diagnosis was confirmed by TEM and PCR. We assembled a VIRRION with ITDs of 200, 100, and 30 nm. This covers the size range of most viruses that commonly cause respiratory infections. Without any sample preparation, we loaded 5 mL of each sample into separate VIRRIONs through a syringe and gently pushed manually. After capture, virus-like particles were observed by SEM on CN×CNT arrays. We extracted nucleic acid on-chip from the VIRRIONs and confirmed by RT-qPCR what viruses were captured. Their Raman spectra fingerprints were determined and recorded. (See FIG. 19). The PCA analysis demonstrated that the Raman spectra could clearly differentiate between the virus strains when converting the data into a low-dimensional (2D) scale. Next, we applied the previously developed machine learning strategy to classify the results of the Raman spectroscopy. The accuracy for identification of the specific viruses was ˜93% using the logistic regression algorithm. Our results indicate that VIRRION can be successfully used to detect specific viruses within several minutes after collecting clinical samples.

After label-free capture and detection, we processed the isolated nucleic acid for NGS and determined that after VIRRION capture, the influenza A and HPIV 3 viruses were enriched. The percentage of virus-specific reads increased from 0.08% to 0.44% for influenza A and from 4.1% to 31.8% for HPIV 3. The percentage of genome covered also increased after enrichment. When considering positions with coverage over 10 counts per million (cpm) for influenza A, the percentage of genome covered increased from 33% to 48% and 57% following the first and second enrichment steps, respectively. The percent genome coverage of HPIV 3 increased from 22% before enrichment to 81% and 77 300% after the first and second enrichment, respectively, when considering positions with coverage over 500 cpm. Their average coverage increased from 8.8× to 15.9× for influenza, and 363× to 2040× for HPIV 3. We were also able to assemble large portions of each viral genome. No influenza A contigs greater than 500 bp were identified following assembly in the unenriched sample. However, 4 contigs matching to the influenza A genome were found in both enrichment steps with an average of 590 bp. The entire genome was assembled in each of the HPIV 3 samples. For influenza A, we also discovered that the virus most closely matched H3N2 strains from the 2016 season. The result of coverage and variant analyses were summarized in FIG. 19. For HPIV 3, we mapped the reads against strain 7N2 (MF973163.1). The NGS results supported that VIRRION can enrich different viruses directly from patient swab samples. Unfortunately, no Rhinovirus related read was detected by NGS after enrichment. From the PCR results, we suspect that this is because the virus titer is extremely low and beyond the detection limit for NGS even after VIRRION enrichment.

As appreciated from the test results, embodiment of the system provide a multifunctional and portable platform for rapid virus capture and sensitive in-situ identification by Raman spectroscopy. The captured viruses are viable and enriched, thus providing effective sample preparation for existing standard methods for virus analysis, including cell culture for virus isolation, immuno-staining, and next-generation sequencing. We also successfully captured and detected different human respiratory viruses from clinical samples using this platform. One of its strengths is that it can perform enrichment in just a few minutes and achieves a sensitivity comparable to that of RT-qPCR with a 70˜90% accuracy. This provides a novel way to overcome the technical barrier in virus surveillance and discovery. These features could also help in virus prediction and outbreak preparedness.

It should be understood that modifications to the embodiments disclosed herein can be made to meet a particular set of design criteria. For instance, the number of components or elements can be any suitable number of each to meet a particular objective. The particular configuration of type of such components or elements can also be adjusted to meet a particular set of design criteria. Therefore, while certain exemplary embodiments of the apparatuses and methods of making and using the same have been discussed and illustrated herein, it is to be distinctly understood that the invention is not limited thereto but may be otherwise variously embodied and practiced within the scope of the following claims.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

What is claimed is:

1. A bioagent capture and identification device, comprising:
a substrate; and
a vertically-aligned carbon nanotube (CN×CNT) array grown on the substrate, the CN×CNT array comprising a plurality of carbon nanotubes having an inter-tubular distance between each carbon nanotube;
wherein the substrate is patterned with a particle precursor; and
wherein at least one nanotube is decorated with an enriching particle.

2. The device of claim 1, further comprising:
a casing having an inlet and an outlet.

3. The device of claim 2, wherein the casing has a casing top, a casing bottom, and casing sides, and the CN×CNT array is vertically orientated with respect to the casing top and the casing bottom.

4. The device of claim 2, wherein the substrate and/or the CN×CNT array is/are bonded to at least a portion of the casing top and/or at least a portion of the casing bottom.

5. The device of claim 1, wherein patterning the substrate with a particle precursor causes a change in the inter-tubular distance between each carbon nanotube.

6. The device of claim 1, wherein the inter-tubular distance between two carbon nanotubes differs from the inter-tubular distance between two other carbon nanotubes.

7. The device of claim 1, wherein the inter-tubular distance between two carbon nanotubes varies along a length of the two carbon nanotubes.

8. The device of claim 1, wherein the particle precursor includes a Fe particle.

9. The device of claim 1, wherein decorating with the enriching particle functionalizes a carbon nanotube.

10. The device of claim 1, wherein the enriching particle includes an Au particle.

11. The device of claim 1, wherein a nanotube of the CN×CNT array is doped with a dopant.

12. The device of claim 11, wherein the dopant is nitrogen.

13. The device of claim 1, wherein at least one carbon nanotube has a herringbone shape.

14. The device of claim 2, wherein a portion of the casing is removable.

15. The device of claim 2, wherein:
the device is a microfluidic platform configured to receive a solution containing a species via the inlet, cause the solution to pass over or through the CN×CNT array, and expel the solution via the outlet; and
the inter-tubular distance between each carbon nanotube is tunable and selected to capture a species based on size.

16. A bioagent capture and identification system, comprising:
a microfluidic device, comprising:
a substrate; and
a vertically-aligned carbon nanotube (CN×CNT) array grown on the substrate, the CN×CNT array comprising a plurality of carbon nanotubes having an inter-tubular distance between each carbon nanotube;
wherein the substrate is patterned with a particle precursor;
wherein at least one nanotube is decorated with an enriching particle; and
wherein the microfluidic device is configured to receive a solution containing a species and capture the species within the inter-tubular distance between two carbon nanotubes based on size; and
a spectrometer configured to generate spectra data of the captured species;
a computer system comprising a computer device and a database, the computer system configured to receive the spectra data and use machine learning techniques to identify the species.

17. The system of claim 16, wherein the spectrometer is a Raman spectrometer.

18. The system of claim 16, wherein the database includes historical spectra data of different species.

19. A method of fabricating a platform for a bioagent capture and identification device, the method comprising:
patterning precursor particles onto the substrate;
growing a vertically-aligned carbon nanotube (CN×CNT) array; and
coating a nanotube of the CN×CNT array with an enriching particle.

20. The method of claim 19, further comprising:
doping a nanotube of the CN×CNT array with a dopant.
